SEQUENCE LISTING (12 sequences)

SEQ ID NO: 1 - protein, 348 aa, Brucella melitensis
MKFARLALMGGIFATVAFTVGPAFARDLTVASWGGNYQDAQREIYFKPFAEKTGKPLLDESWDGGYGVIQSKVKAGSPNWDVVQVEAEELALGCADGLYEKIDWDKVGGKDKFLDSAVNDCGVGAIVWSTAIAYNGDKLKDGPKSWADFWDVKKFPGKRSLRKSAKYTLEFALMADGVDKDDVYDVLSTPEGVDRAFKKLDELKPHIVWWEAGAQPLQLLASDEVVMASAYNGRITGINRSEGKNFKVVWPGSIYAVDSWVILKGAENKDAGLDFIAFASEPEHQVKLPKYVAYGLPNKEAAAKVPEEYAADLPTAKANMKDALALDVDFWIDHSEELTKRFNAWLAQ

SEQ ID NO: 2 - protein, 323 aa, Brucella melitensis
RDLTVASWGGNYQDAQREIYFKPFAEKTGKPLLDESWDGGYGVIQSKVKAGSPNWDVVQVEAEELALGCADGLYEKIDWDKVGGKDKFLDSAVNDCGVGAIVWSTAIAYNGDKLKDGPKSWADFWDVKKFPGKRSLRKSAKYTLEFALMADGVDKDDVYDVLSTPEGVDRAFKKLDELKPHIVWWEAGAQPLQLLASDEVVMASAYNGRITGINRSEGKNFKVVWPGSIYAVDSWVILKGAENKDAGLDFIAFASEPEHQVKLPKYVAYGLPNKEAAAKVPEEYAADLPTAKANMKDALALDVDFWIDHSEELTKRFNAWLAQ

SEQ ID NO: 3 - protein, 367 aa, Brucella melitensis
MGIKSFLLATTVATGFVAAATFSAGAQERVVNIYNWSDYIDDSILKDFTKETGIKVVYDVYDSNEILETKLLAGGSGYDLVVPSGEFLGRQIPAGVFLKLDKDKLPNLKNMWDEISTRAATYDPGNEYSVNYMWGTTGIGYNKAKIKEALGTDTIDSWDVLFDPEKTAKLKDCGIYLLDSASEMLRPALNYLGLDPNSPSPDDLQKAQDLYLKIRPNIRKFHSSEYINALANGDICMAVGYSGDIFQARDRAEKAKQGVEIGYSIPKEGALIWFDQMAIPADAKHVPEALEFMNYMMRPEVAAKASNYVFYANGNKASQKFIDKEILDDPEIYPSDEVMKKLFVPTPYDTKTQRVVTRAWTKIVTGQ

SEQ ID NO: 4 - protein, 341 aa, Brucella melitensis
QERVVNIYNWSDYIDDSILKDFTKETGIKVVYDVYDSNEILETKLLAGGSGYDLVVPSGEFLGRQIPAGVFLKLDKDKLPNLKNMWDEISTRAATYDPGNEYSVNYMWGTTGIGYNKAKIKEALGTDTIDSWDVLFDPEKTAKLKDCGIYLLDSASEMLRPALNYLGLDPNSPSPDDLQKAQDLYLKIRPNIRKFHSSEYINALANGDICMAVGYSGDIFQARDRAEKAKQGVEIGYSIPKEGALIWFDQMAIPADAKHVPEALEFMNYMMRPEVAAKASNYVFYANGNKASQKFIDKEILDDPEIYPSDEVMKKLFVPTPYDTKTQRVVTRAWTKIVTGQ

SEQ ID NO: 5 - DNA, 1047 bp, Brucella melitensis
ttattgcgccagccaggcgttgaaacgcttcgtcagctcttctgaatggtcgatccagaaatcaacatcgagcgcgagggcatccttcatgtttgctttggccgtgggcaggtctgctgcatattcctccggcaccttggctgccgcttccttgttcggcaggccgtaggcaacatatttcggcagcttgacctgatgttcgggctcactggcaaaggcaatgaaatccaggcccgcatccttattttcagcccccttcagaatcacccagctatccaccgcatagatgctgcccggccagacgaccttgaaattcttgccttcggaacggttgatgccagtgatgcggccattataagccgatgccatcaccacctcgtccgacgccagcaattgcaagggctgcgcaccggcttcccaccacacgatatgtggcttcagctcatcgagcttcttgaaggcgcggtccacgccttccggcgtggaaaggacgtcataaacgtcatccttgtcgacgccatcagccataagcgcgaattccagcgtgtatttcgcactcttgcgcagcgatcgcttgcccggaaacttcttcacatcccagaaatccgcccaggatttcggcccatccttcagcttgtcgccattataagctatggcagtggaccagacgatggcgccaaccccgcaatcattgaccgcactgtcgaggaatttgtccttgccgcccaccttgtcccagtcgatcttttcataaagaccatcggcgcaaccgagcgccagctcctccgcctcgacctgaaccacgtcccaattcggcgagccggccttcacttttgactggatgaccccgtaaccgccatcccatgattcatcgagcagcggcttgccggtcttttccgcaaaaggtttgaaatagatttcgcgctgcgcatcctgatagttgccgccccatgacgcgacggtaaggtcgcgcgcgaaggcggggccgacggtgaaggcaacggtagcgaaaattccgcccataagggcaaggcgagcaaacttcat

SEQ ID NO: 6 - DNA, 972 bp, Brucella melitensis
cgcgaccttaccgtcgcgtcatggggcggcaactatcaggatgcgcagcgcgaaatctatttcaaaccttttgcggaaaagaccggcaagccgctgctcgatgaatcatgggatggcggttacggggtcatccagtcaaaagtgaaggccggctcgccgaattgggacgtggttcaggtcgaggcggaggagctggcgctcggttgcgccgatggtctttatgaaaagatcgactgggacaaggtgggcggcaaggacaaattcctcgacagtgcggtcaatgattgcggggttggcgccatcgtctggtccactgccatagcttataatggcgacaagctgaaggatgggccgaaatcctgggcggatttctgggatgtgaagaagtttccgggcaagcgatcgctgcgcaagagtgcgaaatacacgctggaattcgcgcttatggctgatggcgtcgacaaggatgacgtttatgacgtcctttccacgccggaaggcgtggaccgcgccttcaagaagctcgatgagctgaagccacatatcgtgtggtgggaagccggtgcgcagcccttgcaattgctggcgtcggacgaggtggtgatggcatcggcttataatggccgcatcactggcatcaaccgttccgaaggcaagaatttcaaggtcgtctggccgggcagcatctatgcggtggatagctgggtgattctgaagggggctgaaaataaggatgcgggcctggatttcattgcctttgccagtgagcccgaacatcaggtcaagctgccgaaatatgttgcctacggcctgccgaacaaggaagcggcagccaaggtgccggaggaatatgcagcagacctgcccacggccaaagcaaacatgaaggatgccctcgcgctcgatgttgatttctggatcgaccattcagaagagctgacgaagcgtttcaacgcctggctggcgcaataa

SEQ ID NO: 7 - DNA, 1104 bp, Brucella melitensis
atggggatcaaatccttccttctggcaacgaccgttgcgacgggttttgtcgcggctgccacattttccgcaggcgcgcaggagcgggtggtcaatatctataactggtcggattatatcgacgattccatcctcaaggacttcaccaaggagaccgggatcaaggtcgtctacgacgtctatgactccaacgaaattctggaaaccaagcttctggcgggcggcagcggctacgacctcgtggtgccatcgggcgaatttcttgggcgccagattcccgcaggcgtgttcctgaaactcgacaaggacaagctgccgaacctcaagaatatgtgggatgagatttcgacccgtgcggcaacctatgatccgggcaacgaatattccgtcaattacatgtggggcacgaccggcatcggctacaataaggccaagatcaaggaagcgctcggcaccgacacgatcgactcctgggacgtgcttttcgatccggaaaaaacggcaaagctgaaagattgcggcatttacctgcttgattccgccagcgaaatgctgcgtccggcgctgaactatctgggtctcgacccgaactctccgtcgccggacgatttgcagaaggcacaggatctctatctcaagattcgtccgaatatccgcaaattccactcgtcggaatatatcaatgcgctcgccaatggcgatatctgcatggctgtcggctattccggcgatattttccaggcccgcgaccgcgccgaaaaggcgaagcagggggtggagatcggctattcgatcccgaaggaaggcgcgctgatctggtttgaccagatggcgatcccggccgatgccaagcatgtgccggaggcgttggaattcatgaattatatgatgcgcccggaggtggcggcaaaggcgtcgaactatgtgttctatgccaatggcaacaaggcgtcgcagaaattcatcgacaaggaaatcctcgacgacccggaaatctatccgtccgacgaggtgatgaagaagctgttcgtgccgacgccatatgacacgaagacccagcgtgtggtcacacgcgcctggaccaagatcgtcaccggccagtaa

SEQ ID NO: 8 - DNA, 1026 bp, Brucella melitensis
caggagcgggtggtcaatatctataactggtcggattatatcgacgattccatcctcaaggacttcaccaaggagaccgggatcaaggtcgtctacgacgtctatgactccaacgaaattctggaaaccaagcttctggcgggcggcagcggctacgacctcgtggtgccatcgggcgaatttcttgggcgccagattcccgcaggcgtgttcctgaaactcgacaaggacaagctgccgaacctcaagaatatgtgggatgagatttcgacccgtgcggcaacctatgatccgggcaacgaatattccgtcaattacatgtggggcacgaccggcatcggctacaataaggccaagatcaaggaagcgctcggcaccgacacgatcgactcctgggacgtgcttttcgatccggaaaaaacggcaaagctgaaagattgcggcatttacctgcttgattccgccagcgaaatgctgcgtccggcgctgaactatctgggtctcgacccgaactctccgtcgccggacgatttgcagaaggcacaggatctctatctcaagattcgtccgaatatccgcaaattccactcgtcggaatatatcaatgcgctcgccaatggcgatatctgcatggctgtcggctattccggcgatattttccaggcccgcgaccgcgccgaaaaggcgaagcagggggtggagatcggctattcgatcccgaaggaaggcgcgctgatctggtttgaccagatggcgatcccggccgatgccaagcatgtgccggaggcgttggaattcatgaattatatgatgcgcccggaggtggcggcaaaggcgtcgaactatgtgttctatgccaatggcaacaaggcgtcgcagaaattcatcgacaaggaaatcctcgacgacccggaaatctatccgtccgacgaggtgatgaagaagctgttcgtgccgacgccatatgacacgaagacccagcgtgtggtcacacgcgcctggaccaagatcgtcaccggccagtaa

SEQ ID NO: 9 - DNA, 27 bp, Artificial sequence
  Synthetic sequence Primer
cgaattcgcatgcaggagggggtggtc

SEQ ID NO: 10 - DNA, 28 bp, Artificial sequence
  Synthetic sequence Primer
gctcgagcgttactggccggtrgaggat

SEQ ID NO: 11 - DNA, 27 bp, Artificial sequence
  Synthetic sequence Primer
cgaattcgcatgcgcgaccttaccgtc

SEQ ID NO: 12 - DNA, 27 bp, Artificial sequence
  Synthetic sequence Primer
gctcgagcgttattgcgccagccaggc

(19) United States
(12) Patent Application Publication
(10) Pub. No.: US 2011/0033492 A1
(43) Pub. Date: Feb. 10, 2011

(54) VACCINES FOR BRUCELLOSIS

(76) Inventors: Dominic Charles Jenner, Wiltshire (GB); Helen Susan Atkins, Wiltshire (GB)

Correspondence Address: JOHN S. PRATT, ESQ;KILPATRICK STOCKTON, LLP, 1100 PEACHTREE STREET, SUITE 2800, ATLANTA, GA, 30309, US

(21) Appl. No.: 12/809,685

(22) PCT Filed: Dec. 17, 2008
(86) PCT No.: PCT/GB08/04143

§ 371 Date: Sep. 16, 2010

(30) Foreign Application Priority Data
Dec. 19, 2007 (GB) 0724668.9

(51) Int. Cl. A61K 39/02; C12N 5/10; C12N 15/63; A61K 38/16; C07K 14/23; C07K 16/12; C07H 21/04

(52) U.S. Cl. 424/190.1; 435/325; 435/320.1; 514/2.4; 514/21.2; 530/350; 530/387.9; 536/23.7

(57) ABSTRACT

A polypeptide selected from the group consisting of BMEII0923, BMEI0411, a protective fragment of BMEII0923 and BMEI0411 and immunologically active variants of BMEII0923 and BMEIO411, which provides a protective immune response against Brucella infection, for use as a vaccine against Brucellosis. Pharmaceutical compositions comprising these polypeptides and antibodies raised against these polypeptides are described and claimed. Such pharmaceutical compositions are useful in both active and passive vaccination against Brucella infections and may be used to treat Brucella infection or Brucellosis after exposure to the bacteria.

This invention relates to ABC system proteins from Brucella melitensis which are immunogenic and protective against infection by Brucella species. More particularly, this invention relates to recombinant polyamine transport proteins, such as PotD and PotF, and their use in the treatment of brucellosis.

Brucellosis is a bacterial disease caused by members of the gram-negative genus Brucella (including B. melitensis, B. suis, B. neotomae, B. ovis, B. canis and B. abortus). Brucellosis, and particularly that caused by B. melitensis, can affect most species of domestic animals and is transmissible to humans. Sheep and goats, in particular milking breeds of sheep and goats, are the most susceptible to brucellosis but cattle can be affected and the disease may appear in pigs. Infection in animals normally occurs by inhalation and via abraded skin and transmission between species occurs readily.

Human infection usually occurs as a result of contact with infected animals or by ingesting unpasteurized milk or cheese produced from infected animals. In humans the disease is sometimes known as Malta fever or Undulant fever and initial symptoms are flu-like but may lead to acute or chronic infection. Although treatment with antibiotics is possible, long courses of therapy may be required and relapse can occur, even after several years.

There are currently a number of brucellosis vaccines which are licensed for use in livestock, all of which are live attenuated strains of Brucella organisms. S19 and RB51 are live attenuated strains of B. abortus and S2 is a naturally occurring avirulent strain of B. suis. The live attenuated vaccine B. melitensis Rev.1 is currently recommended for the protection of sheep and goats against B. melitensis but there are no vaccines available for preventing brucellosis in humans.

There are a number of issues with the use of live attenuated strains as vaccines. Firstly, since the vaccines are live strains of Brucella, they have the potential to persist within the host and under certain physiological conditions they may revert to pathogens able to cause disease. Secondly, the vaccines trigger immune responses that are currently indistinguishable from field infection thereby rendering them incompatible with test and slaughter based control and eradication policies. Thirdly, and perhaps of most concern to those working with the vaccines, all of the available animal vaccines have the capacity to cause disease in humans.

Several attempts have been made to generate non-living Brucella vaccines to overcome these issues. Various studies have examined cell-free native and recombinant proteins as candidate protective antigens (see, for example, Schurig et al, Veterinary Microbiology 90 (2002) pp 479-496) but with limited success.

More recently, other researchers (Commander et al, Vaccine 25 (2007) pp 43-54) have identified proteins from Brucella melitensis as novel protective antigens and describe DNA vaccines encoding the protective antigens, which displayed good seroreactivity post-vaccination but which will likely require booster inoculations to achieve complete protection. Alternative antigens are therefore sought.

ATP-binding cassette (ABC) transporter systems have provided vaccine targets in several organisms including Salmonella and Streptococcus. The facts that ABC transporter systems constitute one of the largest protein superfamilies, are ubiquitous in nature and that they are responsible for the transport of a wide variety of different molecules across cellular membranes implies that ABC system proteins are involved in many cellular functions including bacterial metabolism and virulence. This, in turn, has prompted the assertion that ABC system proteins may constitute potential vaccine antigens and, in fact, ABC system proteins have been shown to be protective against infection by certain bacteria, including Mycobacterium tuberculosis (Tanghe, A. et al, The Journal of Immunology, 162, pp 1113-1119) and Streptococcus pneumoniae (Brown, J. S. et al, Infection and Immunity, 69, pp 6702-6706.) However, no ABC transport proteins have been shown to be protective against Brucella infection and, within such a vast family of proteins there is no clear indication, firstly, which if any of the proteins may be involved in virulence and, secondly, if any of those proteins will be protective when isolated or expressed recombinantly and administered as a vaccine.

There is clearly a requirement, therefore, to develop new and improved vaccines for brucellosis. Ideally such a vaccine would be a recombinant protein or proteins, or a DNA vaccine encoding such proteins, such that the problems described above relating to live attenuated vaccines can be overcome. A vaccine based on a recombinant protein is particularly advantageous because it can be manufactured without handling of the virulent organism. Also it is likely that a recombinant protein (or mixture of recombinant proteins) will be easier to handle, store and ultimately administer than live vaccines.

The inventors have found that that certain ABC transporter proteins isolated or derived from Brucella melitensis are both immunogenic and protective. The proteins have been cloned, expressed recombinantly, characterised and their potential to induce protective immunity against B. melitensis tested in the mouse model of infection. Both proteins afford protection against B. melitensis infection. The proteins are therefore excellent candidates for a new DNA or protein-based vaccine for preventing brucellosis.

Accordingly, in a first aspect, the present invention provides a polypeptide derived from Brucella melitensis, wherein said polypeptide is selected from the group consisting of BMEII0923, BMEI0411, a protective fragment of BMEII0923, a protective fragment of BMEI0411 and immunologically active variants of BMEII0923 and BMEI0411, which provides a protective immune response against Brucella infection, for use as a vaccine.

It will be understood by the person skilled in the art that the polypeptides above are those which are encoded by the corresponding genes with the locus tags BMEII0923 on chromosome 1 of Brucella melitensis strain 16M and BMEI0411 on chromosome 2 of Brucella melitensis strain 16M, the full chromosome sequences being available as Genbank accession nos NC003317 and NC003318, which are hereby incorporated by reference in their entirety.

As used herein, the expressions PotD and PotF relate to the polypeptides, and the corresponding genes that encode them as appropriate, with locus tags BMEII0923 and BMEI0411, respectively, as described above. These shorthand notations are used for ease of reference only and no limitation to the function of the gene or any corresponding protein is intended by their use herein. In the event of disparity between the sequences indicated by the above locus tags and the sequences listings provided herein, the sequence listings shall take precedence,

As used herein the expression “provides a protective immune response” means that the substance is capable of generating a protective immune response in a host organism such as a mammal, for example a human, to whom it is administered.

As used herein the term “polypeptide” means a sequence of amino acids joined together by peptide bonds. The amino acid sequence of the polypeptide is determined by the sequence of the DNA bases which encode the amino acids of the polypeptide chain. The polypeptides described herein include, but are not limited to, large, full length or “complete” proteins. The terms polypeptide and protein may thus be used interchangeably herein.

As used herein the term “fragment” refers to any portion of the given amino acid sequence of a polypeptide which has substantially the same activity as the complete amino acid sequence. Fragments will suitably comprise at least 5 and preferably at least 10 consecutive amino acids from the basic sequence and does include combinations of such fragments. Fragments will also include truncates of the full amino acid sequence of a full length protein, which are formed by cloning and expressing part of a gene, by cleaving amino acids from either N or C terminus of the full length protein, or both, or by other means which digest or cleave individual amino acids or groups of amino acids from the polypeptide. In order to retain protective activity, fragments will suitably comprise at least one epitopic region. Fragments comprising epitopic regions may be fused together to form a variant. Protective fragments are thus those fragments which provide similar levels of protection to the complete (i.e. non-fragmented) protein on which the fragments is based.

In the context of the present invention the expression “variant” refers to sequences of amino acids which differ from the base sequence from which they are derived in that one or more amino acids within the sequence are substituted for other amino acids. Amino acid substitutions may be regarded as “conservative” where an amino is replaced with a different amino acid with broadly similar properties. “Non-conservative” substitutions are where amino acids are replaced with amino acids of a different type. Broadly speaking, fewer non-conservative substitutions will be possible without altering the biological activity of the polypeptide. Suitably variants will be greater than 79% identical, preferably at least 85% identical, more preferably at least 90% identical, and most preferably at least 95%, 96%, 97%, 98% or 99% identical to the base sequence.

Identity in this instance can be judged for example using the US National Center for Biotechnology Information NCBI's BLAST program (vs. 2.2.12 or later) found at http://balst.ncbi.nlm.nih.gov/Blast.cgi or the algorithm of Lipman-Pearson, with Ktuple:2, gap penalty:4, Gap Length Penalty:12, standard PAM scoring matrix (Lipman, D. J. and Pearson, W. R., Rapid and Sensitive Protein Similarity Searches, Science, 1985, vol. 227, 1435-1441).

As used herein “Brucella infection” means the infection caused by any species of the Brucella genus, which includes, but is not limited to, B. melitensis, B. suis, B. neotomae, B. ovis, B. canis and B. abortus irrespective of being diagnosed as brucellosis.

Particular examples of polypeptides of the invention are those described by SEQ. ID no.1 or SEQ. ID no.2, SEQ. ID no.3 and SEQ. ID no.4. SEQ ID nos.2 is a truncate (fragment) (hereinafter “PotD truncate”) of SEQ. ID no.1, and SEQ. ID no. 4 is a truncate or fragment (hereinafter “PotF truncate”) of SEQ. ID no.2. Both of these fragments have been found to be particularly effective polypeptides. A particularly preferred polypeptide is that described by SEQ. ID no.4.

It will be readily understood by the person skilled in the art that protective fragments and/or immunologically active variants of these proteins can be designed with routine experimentation, for example, large fragments of the polypeptides of differing size may be routinely prepared using for example, PCR technology to amplify and clone part of the gene sequence encoding for the polypeptide. Such variations in the polypeptide, as a result of for example the use of different PCR primers, are clearly intended to fall within the scope of the present invention.

Where the polypeptide of the invention is an immunologically active variant of the polypeptides described above, it is preferred that the polypeptide has at least 80% sequence identity to said polypeptide. It is more preferred that the variant has at least 90% sequence identity and even more preferred that the variant has at least 95%, 97% or 99% sequence identity to said polypeptide.

The polypeptides of the present invention may be isolated from live strains of Brucella melitensis or may alternatively and conveniently be prepared using standard cloning and expression technology which is well known in the art. For example, the genes that encode the proteins, or the fragments of the genes that encode specific peptide regions of the corresponding proteins are amplified. The amplified gene fragments may then be cloned into an expression vector to produce a recombinant plasmid, which may, in turn be transformed into a host cell. Host cells may be prokaryotic or eukaryotic cells but it is preferred that the host cell is a prokaryotic cell, such as Escherichia coli.

As is well understood in the art, such expression systems may include standard expression or purification tags, which may or may not be cleaved as necessary prior to use.

The polypeptides are useful as vaccines against brucellosis. Consequently, according to a second aspect of the invention, there is provided a pharmaceutical composition comprising a polypeptide as described above, together with an adjuvant.

The pharmaceutical composition will conveniently comprise a pharmaceutically acceptable carrier or excipient. Suitable excipients and carriers will be known to those skilled in the art. These may include solid or liquid carriers. Suitable liquid carriers include water or saline. The polypeptides of the composition may be formulated into an emulsion or alternatively they may be formulated in, or together with, biodegradable microspheres or liposomes.

Many adjuvants will be suitable components of the pharmaceutical composition provided that the adjuvant stimulates an immune response in a host to whom the composition is administered. Particularly suitable adjuvants include, but are not limited to, ISCOMS, CpGs, Alhydrogel, MPL+TDM and Freunds Incomplete Adjuvant.

In a preferred embodiment, the composition further comprises an additional immunogenic polypeptide which provides at least some protective effect against Brucella. The additional polypeptide may be a second one selected from the polypeptides described herein, or it could be an existing protective antigen for Brucella, such as the Brucella melitensis Invasion Protein B, lalB (BMEI1584). It will be understood by the person skilled in the art that more than one additional immunogenic polypeptide can be used in the composition and examples of further suitable polypeptides include others described in US Patent application no 2007/0224257, the contents of which are incorporated herein by reference.

According to a third aspect of the invention there is provided a nucleic acid which encodes any of the ABC transporter proteins described herein, for use as a vaccine.

Such nucleic acids can be used to produce the polypeptide described above, for use as a vaccine. For instance they can be incorporated into an expression vector, which is used to transform an expression host such as a prokaryotic or eukaryotic cell, and in particular is a prokaryotic cell such as E. coli using recombinant DNA technology as would be understood in the art. Such vectors, cells and expression methods form further aspects of the invention.

Preferred nucleic acids are SEQ. ID no.5, SEQ. ID no 6, SEQ. ID no.7 and SEQ. ID no.8. It will be understood by those skilled in the art that the nucleic acids of the present invention may optionally include nucleic acid sequences which encode for expression and/or purification tags.

Alternatively, the nucleic acids can be used in “live” or “DNA” vaccines to deliver the polypeptide to the host animal. It is preferred that DNA vaccines will be delivered in a pharmaceutical composition comprising a plasmid vector which comprises the nucleic acid of the invention. Thus, a pharmaceutical composition comprising a plasmid vector which comprises the nucleic acid of the invention forms a fourth aspect of the invention.

All of the above mentioned products are useful for use in the treatment of infection by Brucella species. Thus use of the proteins and nucleic acids described above in the treatment of brucellosis forms a fifth aspect of the invention. Particularly, the products are useful in the treatment of infection by Brucella melitensis or Brucella abortus.

In a further aspect of the invention, there is provided an antibody raised against the ABC transporter polypeptides described above, or a binding fragment of such an antibody. Since the polypeptides are immunogenic, they are capable of inducing an immune response in a mammal to which they are administered. Antibodies may be raised in-vivo against the complete polypeptides, or they may be raised against suitable epitopic fragments of the polypeptides using conventional methods.

Binding fragments include Fab, F(ab′)₂, Fc and Fc′.

Antibodies may be polyclonal or monoclonal. Hybridoma cell lines which generate monoclonal antibodies of this type form a further aspect of the invention.

Antibodies themselves, for example in the form of sera comprising such antibodies may be useful in the passive vaccination and/or treatment of compromised individuals.

According to a yet further aspect of the invention there is provided a method of protecting a human or animal body from the effects of infection with Brucella species comprising administering to the body a vaccine comprising a nucleic acid, a polypeptide or a pharmaceutical composition as described above. The nucleic acid, polypeptide or composition is capable of inducing a protective immune response in a mammal to which it is administered and the ability to elicit an effective immune response may be provided by an epitopic fragment or a variant of said protein (or by the nucleic acid encoding for such an epitopic fragment or variant). Particular examples of suitable proteins include SEQ ID nos. 1, 2, 3 and 4 and examples of suitable nucleic acids include SEQ. ID nos. 5, 6, 7 and 8.

The polypeptides, nucleic acid and pharmaceutical compositions described herein are useful in both active and passive vaccination against Brucella infections and may be used to treat Brucella infection or Brucellosis after exposure to the bacteria.

The vaccine may be administered prophylactically to those at risk of exposure to Brucella or may be administered as a therapeutic treatment to persons who have already been exposed to Brucella melitensis, although it will be understood that it may be advantageous in particular circumstances to administer antibodies as described above together with or instead of the vaccine, for example, antibodies may be administered prophylactically to ensure rapid immunity (particularly if a gap exists between administration of sub-unit vaccine and the patient acquiring full immunity) and/or they may be administered as a post-exposure therapy to provide rapid treatment of infection after exposure. The route of administration of the vaccine may be varied depending on the formulation of the polypeptides of the composition. The composition may be suited to parenteral administration (including intramuscular, subcutaneous, intradermal, intraperitoneal and intravenous administration) but may also be formulated for non-parenteral administration (including intranasal, inhalation, oral, buccal, epidermal, transcutaneous, ocular-topical, vaginal, rectal administration).

The invention will now be described by way of non-limiting examples, with reference to the accompanying drawings in which:

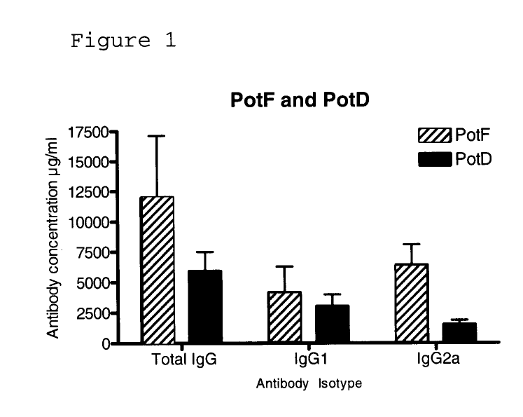

FIG. 1 shows the antibody response to PotD and PotF truncates (according to SEQ ID nos 2 and 4) when administered as recombinant protein vaccines to mice prior to exposure to Brucella melitensis. Naïve mouse sera was tested and found to be below assay detection limits for all three groups.

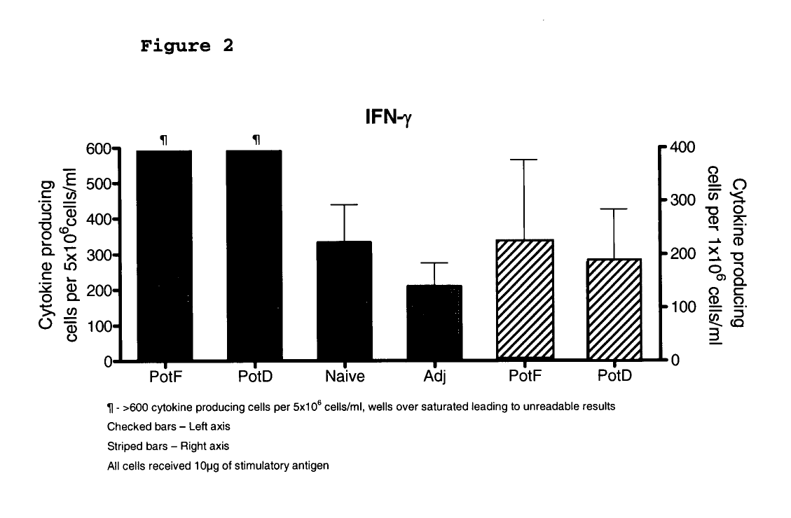

FIG. 2 shows the IFN-γ cytokine responses to the same PotD and PotF vaccine candidates when administered in the same way as above.

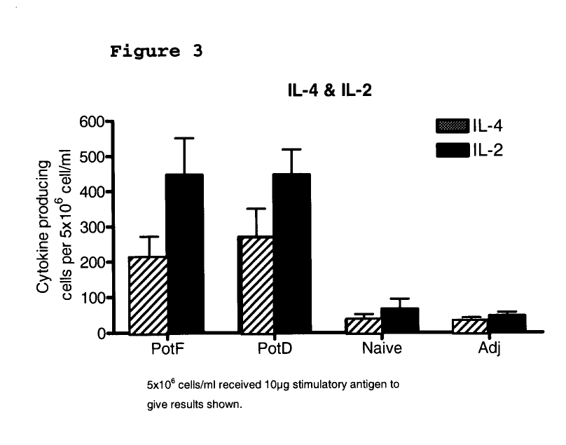

FIG. 3 shows the IL-4 and IL-2 cytokine responses to PotD and PotF vaccine candidates as a result of the same experiment.

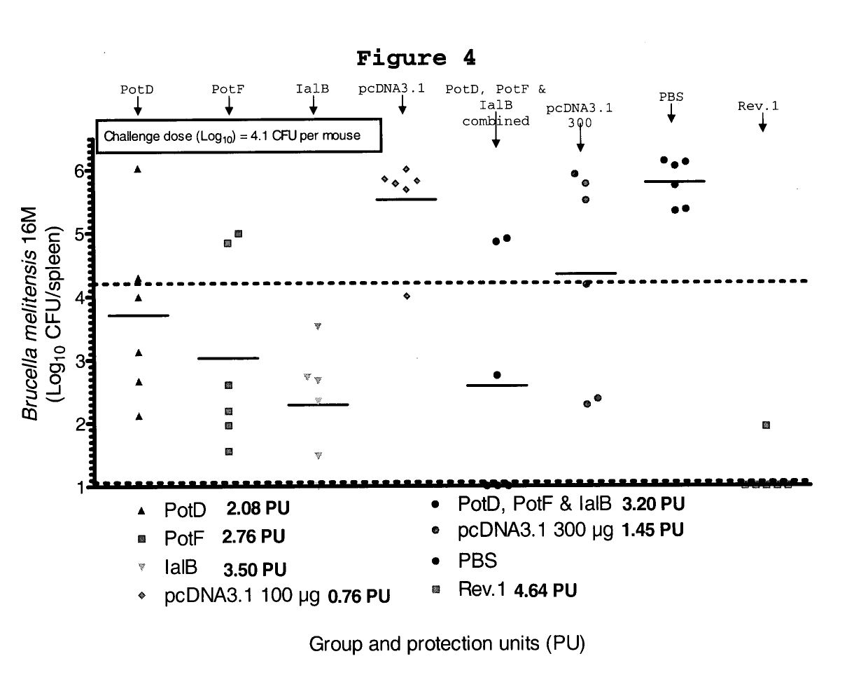

FIG. 4 shows the protection elicited by DNA vaccines encoding truncated PotD, PotF (i.e. administration of DNA comprising SEQ ID nos 6 and 8, respectively) and lalB proteins.

### EXAMPLE 1

### Preparation of and Immunisation with Protective Polypeptides; Challenge with Brucella melitensis in the Mouse Model of Infection

### Bacterial Strains, Media and Culture Conditions

Escherichia coli TOP10F′ cells (Invitrogen Ltd. Paisley, UK) were used for cloning experiments and E. coli BL21 (DE3) pLysS cells (Invitrogen Ltd. Paisley, UK) were used for protein expression studies. B. melitensis 16M DNA was used for all protein production and B. melitensis 16M was used to challenge mice. E. coli strains were cultured in Luria broth (L-broth) shaking at 180 rpm or on Luria agar (L-agar) plates at ACDP containment level 2 conditions. L-broth consisted of 1% (w/v) Bacto tryptone, 0.5% (w/V) Bacto yeast extract, and 0.5% (w/v) sodium chloride in distilled water. L-agar was prepared by the addition of 2% (w/v) Bacto agar to L-broth. Luria media was supplemented with ampicillin and/or chloroamphenicol when appropriate. B. melitensis strains were cultured on nutrient agar or statically in nutrient broth at ACDP containment level 3 conditions. All bacteria were cultured at 37° C. for 18 hours unless otherwise stated.

### Cloning and Expression

Amino acid sequences of the selected proteins were analysed using two web based programs called TMHMM v 2.0 (http://www.cbs.dtu.dk/servicesaMHMM-2.0/) and SignalP (http://www.cbs.dtu.dk/services/SignalP/) to identify regions encoding transmembrane domains and signal peptides respectively. Oligonucleotide primers were designed to amplify DNA sequence encoding the non-membrane/non-signal peptide regions of the proteins. These gene truncates were amplified from B. melitensis 16M genomic DNA by PCR using oligonucleotide primers listed in Table 1. The amplified gene fragments were cloned into the pCR®T7/NT-TOPO expression vector (Invitrogen Ltd. Paisley, UK), and the recombinant plasmids were transformed into Escherichia coli TOP10F′ cells (Invitrogen Ltd. Paisley, UK), according to manufacturer's instructions. Sequencing reactions were performed by Lark Technologies (Takeley, Essex, UK) to ensure correct cloning and the incorporation of a N terminal His₆-tag. Subsequently, E. coli BL21 (DE3) pLysS cells (Invitrogen Ltd. Paisley, UK) were transformed with the recombinant plasmids, according to manufacturer's instructions. Transformants were grown in culture and the addition of 1 mM Isopropyl-β-D-thiogalactoside (IPTG) was used to induce the expression of protein from the recombinant plasmids. Protein expression was confirmed by SDS-PAGE performed using Phastsystem gels and apparatus (Amersham Biosciences Chalfont-St Giles, UK) and visualised by PhastGel Blue R (Amersham Biosciences Chalfont-St Giles, UK). Expressed proteins were transferred to a membrane for detection by Western blot using transfer apparatus of the Phastsystem with the proteins detected with anti-His antibody and developed using 3,3′-Daminobenzidine (DAB) (Sigma-Aldrich Co. Ltd., Poole, UK).

TABLE 1
Oligonucleotide sequences used to amplify target genes in
the Brucella melitensis 16 M genome for cloning purposes.

| | Oligonucleotide sequence and built | |
|---|---|---|
| Primer | in restriction site (5′ to 3′) | Description |
| BM/PotF/ | C GAATTC GC ATG CAG GAG GGG GTG GTC | PotF, forward |
| C2 | EcoR1 | primer, |
| | | truncated |
| BM/PotF/ | G CTCGAG CG TTA CTG GCC GGTR GAG GAT | PotF, reverse |
| C3 | Xho1 | primer, |
| | | truncated |
| BM/PotD/ | C GAATTC GC ATG CGC GAC CTT ACC GTC | PotD, forward |
| C2 | EcoR1 | primer, |
| | | truncated |
| BM/PotD/ | G CTCGAG CG TTA TTG CGC CAG CCA GGC | PotD, reverse |
| C3 | Xho1 | primer, |
| | | truncated |

Key to Table 1:
1) Underlined text denotes a restriction enzyme site, with the specific enzyme noted below the underlined text.
2) Bold text denotes a start codon
3) Italic text denotes a stop codon

### Protein Purification

To produce purified protein, E. coli BL21 (DE3) pLysS cells expressing recombinant protein were centrifuged at 10,000 rpm for 15 min. Pelleted cells were resuspended in Phosphate buffered saline (PBS) plus 300 μg ml⁻¹ DNAase I and Complete EDTA-free protease inhibitor (Roche Diagnostics Ltd Lewes, UK). Cell suspensions were sonicated at 10 microns in 3×30 sec pulses and lysed cells were centrifuged at 15,000 rpm for 20 min. The supernatants were removed and sterilised through a 0.22 μm filter. Immobilised Metal Affinity Chromatography (IMAC) was carried out under the control of AKTA Fast Protein Liquid Chromatography (FPLC) (Amersham Biosciences Chalfont-St Giles, UK) and using Unicorn software version 4.0 (Amersham Biosciences Chalfont-St Giles, UK). Filtered supernatant was applied to a 1 ml His-Trap column (Amersham Biosciences Chalfont-St Giles, UK) with Start buffer (50 mM tris, 750 mM NaCl pH 7.5) and wash fractions were collected, according to manufacturers instructions. Recombinant His-tagged protein was eluted with elution buffer (50 mM Tris, 750 mM NaCl, 500 mM Imidazole, pH 7.5) and was collected in 1 ml fractions. Collected fractions were analysed for purified protein by SDS-PAGE. Fractions containing purified protein were dialysed in PBS to remove imidazole and purified protein was stored at −80° C.

Immunisation and Challenge with B. melitensis 16M

Purified proteins were produced for evaluation as candidate vaccines against B. melitensis. The concentrations of the proteins were ascertained by BCA assay (Pierce Biotechnologies) and purity was evaluated by SDS-PAGE and staining with PhastGel Blue R. 10 μg of protein was prepared for immunisation mixed with 12.5 μg ISCOMs and 6.25 μg CpG adjuvant dissolved in 100 μl PBS. Groups of 6 female BALB/c mice (6 to 10 weeks old) were used. Proteins were delivered by intramuscular (i.m.) injection on days 0, 21 and 42. Blood was taken via the tail vein from each mouse on day 58 Blood samples were allowed to clot for 2-24 h at 4° C. and the centrifuged for 10 min at 13,000 rpm and the serum was stored at −20° C. until use. On day 70 the mice were challenged with approximately 4×10⁴ colony forming units (cfu) of B. melitensis 16M via the i.p. route. Groups of age matched mice were left untreated, or given adjuvant only or B. melitensis live attenuated vaccine Rev. 1 as controls. 15 days after challenge mice were culled and spleens removed mascerated and serially diluted in 0.1M PBS, each dilution was plated out in triplicate. Plates were incubated at 37° C. for 7 days and bacterial colonies counted and back calculated to obtain actual bacterial numbers. Data is presented as Log₁₀ Colony Forming Units (Log₁₀ CFU).

### Antibody Response Analysis

The protein-specific IgG1 or IgG2a responses in blood samples removed from mice prior to challenge were determined by enzyme-linked immunosorbant assay. Briefly, microtitre plates were coated with 5 μg ml⁻¹ of recombinant protein in PBS over night. Three columns on each plate were coated with anti-IgG1 (fab) or anti-IgG2a (fab) in order to produce a standard curve for quantification of IgG1 or IgG2a concentration respectively. The plates were blocked using 2% (w/v) skimmed milk powder in PBS (Blotto) for 1 h at 37° C. Plates were washed and serum samples diluted in blotto were serially diluted 1:2 down the plate in duplicate and incubated for 1 h at 37° C. IgG1 or IgG2a isotype standards were also serially diluted 1:2 down the plate wells coated with anti-IgG1 (fab) or anti-IgG2a (fab) respectively. Three washes with PBS plus 0.05% (v/v) Tween 20 were carried out between each step. Goat anti-mouse IgG1 or IgG2a horseradish peroxidase conjugates diluted in blotto were added and after a further 1 h at 37° C., bound antibody was visualised by adding ABTS substrate. The plates were incubated at room temperature for 20 min before measuring OD₄₁₄ₙₘ. Serum antibody concentration was calculated in μg/ml using ELISA for Windows software (Centre for Disease Control, Atlanta, Ga.).

Antibody responses (FIG. 1) were measured via ELISA and three measurements were made; total specific IgG; IgG1a well characterised marker of Th2 type immune response; and IgG2a a well characterised marker of Th1 immune response. Analysis of the antibody responses show that both of these proteins are immunogenic and that they are recognised by and reacted to by the host immune system. The total IgG concentrations produced show that PotF induces a slightly larger antibody-mediated response than PotD. Although there are small fluctuations between antibody isotype there are no statistically signification bias results towards either a Th1 or Th2 response from either protein.

### Cytokine Response Analysis

Studies were conducted to look at the cytokine response generated by the proteins of interest. Mice were immunised as above and instead of being challenged on day 70 mice spleens were removed and used for cytokine analysis in elispot assays.

Elispot assay kits were purchased from BD bioscience (San Diego, Calif. USA) and used as manufacturers instructions. Briefly, 96 well sterile assay plates were coated in an anti-cytokine antibody diluted in PBS to a concentration of 5 μg/ml, 100 μl was added to each well. Plates were then stored at 4° C. for at least 12 hrs but no more than 4 days before use.

Antigens were diluted in RMPI culture media (RPMI 1640 containing 10% fetal bovine serum (FBS, Sigma, Poole, UK) and 1% penicillin/streptomycin/L-glutamine solution (Gibco, Paisley, UK) to the following concentrations 20, 2; 0.2 μg/ml, this takes into account they will be further diluted by a 1/2 on the plate and so final antigen concentrations will be 10, 1, 0.1 μg/ml. Controls on the plates consisted of cells incubated with no stimulatory antigen, and positive control was concanavalin A (conA—Sigma, Poole UK) which was used at a concentration of 1 μg/ml.

Plates were washed manually once with RMPI culture media and then blocked with 100 μl of RPMI culture media. Plates were incubated at room temperature for 2 hrs. During this period mouse spleens were prepared as follows. Spleens were removed from immunised mice, homogenised through a nylon sieve and suspended in 5 ml of RMPI culture media. 5 ml of red cell lysis buffer (Sigma, Poole, UK) was added and spleen homogenates were incubated at room temperature for 1 minute before being spun down at 1200 rpm for 10 minutes. Supernatants were discarded and pellets resuspended in between 2-5 ml of RPMI culture media depending on pellet size. Once resuspended cells were counted using a haemocytometer (Sigma, Poole UK). Cells were mixed in a 1:1 ratio of trypan blue (Sigma, Poole, UK) and live cells (clear) were counted under a light microscope. Cell density was calculated and cells were resuspended to concentrations of 1×10⁶ cells/ml and 5×10⁶ cells/ml. Cells (100 μl per well) and antigen (100 μl) were plated out and plates were incubated at 37° C.+5% CO₂ and relative humidity for 16-20 hrs.

Wash steps were carried out manually with PBS+0.05% Tween20 (PBST), PBS or dH₂O. Cell and antigen mix was aspirated from the well and plates were washed 2×dH₂O and then 3×PBST. Biotinylated detection antibody was diluted to 2 μg/ml in PBS+10% FBS and 100 μl was added to each well. Plates were incubated at room temperature for 2 hrs. Plates were washed 3×PBST and enzyme conjugate (Streptavidin-HRP) was added (100 μl/well) at a dilution of 1:100. Plates were incubated at room temperature for 1 hr. Plates were washed 4×PBST and 2×PBS and 100 μl AEC substrate reagent (BD Biosciences, San Diego, Calif. USA) was made and used according to manufacturers instructions. Plates were left at room temperature to develop for between 5-20 minutes, before being stopped by aspirating off the substrate solution and addition of 100 μl dH₂O. Plate were left to air dry for 16-20 hrs before being read on an Elispot Reader System ELRO4 (Advanced Imaging Devices, Strasberg, Germany).

Blank subtractions of cells with no stimulatory antigen were deducted from cells with stimulatory antigen and analysis of the data was performed using Microsoft® Excel and Graphpad Prism 4 (Graphpad software Inc, CA, USA).

Elispot assays were used to assess the amount of cytokines that are produced in response to the protein immunisations. Cytokines tested for were Interleukin-4 (IL-4), which is secreted from T helper cells and act upon resting and active B cells to up MHC class II expression, on this activation B cells proliferate, differentiate, and increase antibody production. IL-4 is a good marker of a Th2 type immune response and is generally not a cytokine that is required for clearance of an intracellular pathogen and therefore provides an indication of the level of a Th2 response. Interleukin-2 (IL-2), is secreted by T helper cells and acts upon T cells and natural killer cells. It increases proliferation and stimulates T cells to produce their own IL-2. It has also been shown that IL-2 is only one of two cytokines that can clear Brucella infection in vivo and in vitro. Finally, Interferon-γ (IFN-γ) is considered because it is important in Brucella immunology because it has been shown that knockout IFN-γ mice die from the brucellosis whereas in normal mice the infection in cleared in 6 weeks. Secreted by CD4, CD8 T cells and activated natural killer cells, the main function of IFN-γ is to activate macrophages and to increase expression of MHC class I on antigen presenting cells. Once activated macrophages become more capable of killing intracellular pathogens and have an increased antigen presentation capability. Both IL-2 and IFN-γ are markers of the Th1 type immune response and are required for Brucella clearance in mice.

IFN-γ cytokine analysis (FIG. 2) was performed using 1×10⁶ cells/ml with 10 μg stimulatory antigen to ensure levels within the assay detection limits. IL-4 and IL-2 analysis (FIG. 3) were both performed using 5×10⁶ cells/ml and 10 μg stimulatory antigen. Comparing the amount of IFN-γ produced (FIG. 2) between immunised mice and naïve/adjuvant control mice shows that at the 5×10⁶ cell density there is a large amount of IFN-γ produced, comparing this to the naïve and adjuvant only controls there is a higher proportion of IFN-γ produced by the immunised mice. The IFN-γ data that has been produced using 1×10⁶ cell/ml although slightly lower than the control data shows that comparative data can be produced from a cell population five times less dense.

It can be seen that both the PotD and PotF immunised mice have produced larger quantities of both IL-2 and IL-4 when compared to the naïve and adjuvant only mice. This coupled with the IFN-γ data means that there is a good response from the Th1 branch of the immune system. There is no statistical significance to the difference between the IL-4 and IL-2 data sets, which demonstrates that a balanced immune response is generated by the proteins.

Protection Afforded Against B. melitensis 16M

Mice were given 3 doses, 3 weeks apart of 10 μg PotD or PotF+12.5 μg ISCOMs and 6.25 μg CpG, control mice were given 100 μl PBS, Adjuvant only in same quantities as stated above or 1 dose of between 1.5×10⁵-3.5×10⁵ CFU of B. melitensis Rev.1. 30 days post vaccination mice were challenged with approximately 4×10⁴ B. melitensis 16M, 15 days later mouse spleens were harvested homogenised, serially diluted and plated out in triplicate. Plates were counted and CFU were calculated for each mouse spleen. Data is averages of at least five mice and is presented as Log₁₀ CFU±standard deviation (Table 2).

TABLE 2
Log10 challenge data from PotF and PotD trial

| | Log₁₀ CFU/spleen | Protection |
|---|---|---|
| Vaccine | (mean ± SD) | Units (PU) |
| PotF + Adj | 2.56 ± 2.0* | 2.46 |
| PotD + Adj | 1.41 ± 0.814* | 3.61 |
| Adj only | 2.51 ± 1.038* | 2.51 |
| PBS | 5.02 ± 0.272^{$} | 0.00 |
| Rev. 1 | 1.92 ± 0.660* | 3.10 |

*Statistically significant reduction in bacterial load when compared to PBS
^{$}Statistically significant increase bacterial load when compared to Rev.1 Log10 challenge dose = 4.114
Adj = Adjuvant = 12.5 μg ISCOMs & 6.25 μg CpG
Protection Units calculated by [mean Log₁₀ CFU PBS - mean Log1₀ CFU vaccine candidates]

Rev.1 and PBS immunised mice were used as experimental control mice. PotF has a statistically significant reduction in bacterial load when compared to PBS (P<0.05). With protective units of 2.46 it shows good potential for future use. PotD again has a statistically lower bacterial load than PBS (P<0.001). With protection units of 3.61 this protein may be preferred as a vaccine candidate against B. melitensis 16M and is immunisation with PotD provides better protection than Rev.1, the current licensed vaccine, using this animal model. The adjuvant only control does show some control of infection and also has a statistically lower bacterial load than the PBS control (P<0.05). Whilst it is not clear why this response was observed, it is not wholly unusual for some immune response to be produced by adjuvants, particularly CPGs. It is clear, however, that both PotD and PotF when administered together with an adjuvant (i.e. ISCOM+CpG) demonstrate excellent protective properties against B. melitensis 16M challenge.

### EXAMPLE 2

### DNA Vaccines Encoding PotD, PotF and lalB Tested in the Brucella Murine Model of Infection

### Construction of DNA Vaccines

PotD and PotF DNA vaccines were constructed by Geneart AG (Regensburg, Germany), truncated PotD and PotF genes (SEQ. ID nos 6 and 8) were inserted into the pcDNA3.1 DNA vector (Invitrogen Ltd, Paisley, UK), which is commonly used in the Brucella field (see, for example, Commander, N. J., Spencer, S. A., Wren, B. W., and MacMillan, A. P., 2007, “The identification of two protective DNA vaccines from a panel of five plasmid constructs encoding Brucella melitensis 16M genes” Vaccine, 25(1), 43-54. DNA vaccines were purified using Qiagen endotoxin free giga prep kits according to manufacturer's instructions. DNA vaccine concentration was determined using A₂₆₀-A₂₈₀ optical density readings and purity was assessed using A₂₆₀-A₂₈₀ ratios, which had to fall between 1.8 and 2.0 otherwise batches were discarded.

### Vaccination Schedule

Groups of six (6-8 week old) Balb/C mice were immunised with four doses of 100 μl DNA vaccine encoding PotD (SEQ. ID no.5), PotF (SEQ. ID no.6), or lalB, doses were administered at three week intervals. The DNA vaccine encoding lalB (BMEI1584) was chosen as positive control since it has previously shown to be protective (see Commander, N. J. et al, supra for nucleic acid sequences and protection data; the contents of which are hereby incorporated by reference). One group of six mice was also inoculated with a combination of 300 μg of DNA vaccine comprising of 100 μg of each DNA vaccine. A group of mice immunised with a single dose of 2×10⁵ CFU Brucella melitensis Rev.1 was included as a further positive control. Negative controls consisting of three groups of mice immunised with 100 μl PBS, 100 μg pcDNA3.1 (blank vector) or 300 μg pcDNA3.1 were also included in the study. Thirty days after the final inoculation mice were challenged with approximately 1×10⁴ CFU B. melitensis 16M, fifteen days after challenge mice were culled and their spleens removed for splenic colonisation assays. Results are expressed as average B. melitensis 16M Log₁₀ CFU/spleen and as protective units (PU=Log₁₀ CFU PBS immunised mice−mean Log₁₀ CFU vaccine candidates).

### Results

The results of the challenge experiment are shown in FIG. 4, in which the horizontal lines represent the average growth of bacteria after immunisation and challenge. The data sets from left to right relate to the following groups of DNA vaccines, or controls as appropriate:
(1) PotD DNA 2.08 PU
(2) PotF DNA 2.76 PU
(3) lalB DNA 3.50 PU
(4) pcDNA 3.1 (100 micrograms) 0.76 PU
(5) Combined PotD, PotF and lalB DNA 3.20 PU
(6) pcDNA 3.1 (300 micrograms) 1.45 PU
(7) PBS blank
(8) Rev.1 4.64 PU

The results shown in FIG. 4 indicate that, as expected, the DNA vaccine encoding lalB (3.50 PU) elicits good protection. This is consistent with published data regarding this vaccine candidate (Commander et al, supra). However, it is also demonstrated that both PotD and PotF induce a protective immune response as they reduced Brucella growth by at least 2 logs when compared to the PBS and pcDNA3.1 100 μg (negative) controls. The results observed from immunisation with the potF DNA vaccine candidate indicate a significant drop in Brucella spleen loads when compared to the PBS and pcDNA3.1 100 μg (negative) controls and whilst the results from the PotD immunisation show a less significant decrease, it is apparent that a large amount of variation exists within this data set. Certainly, reduced bacterial growth was observed with more than half of the samples within this set, indicating that the PotD DNA has good potential as a vaccine. Inoculating mice with a combination of PotD, PotF and lalB DNA vaccines did not appear to increase the overall effectiveness of protection over inoculation with lalB alone but the combined DNA vaccine does show a significant decrease in the Brucella spleen load from mice inoculated with PBS, which indicates that there may be some benefits in combined administration. This data provides evidence that both PotD and PotF could be administered as DNA vaccine. In both cases, protection was afforded, in mice, against a Brucella infection. PotF and PotD could also be used in combination with IaIB in combined DNA vaccination, without deleterious effects.

### Sequences:

SEQ. ID no. 1:
Amino acid sequence of “PotD” protein
(BMEII0923)- 348 a.a.
MKFARLALMGGIFATVAFTVGPAFARDLTVASWGGNYQDAQREIYFKPFA
EKTGKPLLDESWDGGYGVIQSKVKAGSPNWDVVQVEAEELALGCADGLYE
KIDWDKVGGKDKFLDSAVNDCGVGAIVWSTAIAYNGDKLKDGPKSWADFW
DVKKFPGKRSLRKSAKYTLEFALMADGVDKDDVYDVLSTPEGVDRAFKKL
DELKPHIVWWEAGAQPLQLLASDEVVMASAYNGRITGINRSEGKNFKVVW
PGSIYAVDSWVILKGAENKDAGLDFIAFASEPEHQVKLPKYVAYGLPNKE
AAAKVPEEYAADLPTAKANMKDALALDVDFWIDHSEELTKRFNAWLAQ
SEQ. ID no.2:
Amino acid sequence of “PotD truncate” protein-
323 a.a.
RDLTVASWGGNYQDAQREIYFKPFAEKTGKPLLDESWDGGYGVIQSKVKA
GSPNWDVVQVEAEELALGCADGLYEKIDWDKVGGKDKFLDSAVNDCGVGA
IVWSTAIAYNGDKLKDGPKSWADFWDVKKFPGKRSLRKSAKYTLEFALMA
DGVDKDDVYDVLSTPEGVDRAFKKLDELKPHIVWWEAGAQPLQLLASDEV
VMASAYNGRITGINRSEGKNFKVVWPGSIYAVDSWVILKGAENKDAGLDF
IAFASEPEHQVKLPKYVAYGLPNKEAAAKVPEEYAADLPTAKANMKDALA
LDVDFWIDHSEELTKRFNAWLAQ
SEQ. ID no. 3:
Amino acid sequence of “PotF” protein
(BMEI0411)- 367 a.a.
MGIKSFLLATTVATGFVAAATFSAGAQERVVNIYNWSDYIDDSILKDFTK
ETGIKVVYDVYDSNEILETKLLAGGSGYDLVVPSGEFLGRQIPAGVFLKL
DKDKLPNLKNMWDEISTRAATYDPGNEYSVNYMWGTTGIGYNKAKIKEAL
GTDTIDSWDVLFDPEKTAKLKDCGIYLLDSASEMLRPALNYLGLDPNSPS
PDDLQKAQDLYLKIRPNIRKFHSSEYINALANGDICMAVGYSGDIFQARD
RAEKAKQGVEIGYSIPKEGALIWFDQMAIPADAKHVPEALEFMNYMMRPE
VAAKASNYVFYANGNKASQKFIDKEILDDPEIYPSDEVMKKLFVPTPYDT
KTQRVVTRAWTKIVTGQ
SEQ. ID no. 4:
Amino acid sequence of “PotF truncate” protein
(BMEI0411)- 341 a.a.
QERVVNIYNWSDYIDDSILKDFTKETGIKVVYDVYDSNEILETKLLAGGS
GYDLVVPSGEFLGRQIPAGVFLKLDKDKLPNLKNMWDEISTRAATYDPGN
EYSVNYMWGTTGIGYNKAKIKEALGTDTIDSWDVLFDPEKTAKLKDCGIY
LLDSASEMLRPALNYLGLDPNSPSPDDLQKAQDLYLKIRPNIRKFHSSEY
INALANGDICMAVGYSGDIFQARDRAEKAKQGVEIGYSIPKEGALIWFDQ
MAIPADAKHVPEALEFMNYMMRPEVAAKASNYVFYANGNKASQKFIDKEI
LDDPEIYPSDEVMKKLFVPTPYDTKTQRVVTRAWTKIVTGQ
SEQ. ID no. 5:
Nucleic Acid Sequence encoding “PotD” protein
(BMEII0923)- 1047 bp
TTATTGCGCCAGCCAGGCGTTGAAACGCTTCGTCAGCTCTTCTGAATGGT
CGATCCAGAAATCAACATCGAGCGCGAGGGCATCCTTCATGTTTGCTTTG
GCCGTGGGCAGGTCTGCTGCATATTCCTCCGGCACCTTGGCTGCCGCTTC
CTTGTTCGGCAGGCCGTAGGCAACATATTTCGGCAGCTTGACCTGATGTT
CGGGCTCACTGGCAAAGGCAATGAAATCCAGGCCCGCATCCTTATTTTCA
GCCCCCTTCAGAATCACCCAGCTATCCACCGCATAGATGCTGCCCGGCCA
GACGACCTTGAAATTCTTGCCTTCGGAACGGTTGATGCCAGTGATGCGGC
CATTATAAGCCGATGCCATCACCACCTCGTCCGACGCCAGCAATTGCAAG
GGCTGCGCACCGGCTTCCCACCACACGATATGTGGCTTCAGCTCATCGAG
CTTCTTGAAGGCGCGGTCCACGCCTTCCGGCGTGGAAAGGACGTCATAAA
CGTCATCCTTGTCGACGCCATCAGCCATAAGCGCGAATTCCAGCGTGTAT
TTCGCACTCTTGCGCAGCGATCGCTTGCCCGGAAACTTCTTCACATCCCA
GAAATCCGCCCAGGATTTCGGCCCATCCTTCAGCTTGTCGCCATTATAAG
CTATGGCAGTGGACCAGACGATGGCGCCAACCCCGCAATCATTGACCGCA
CTGTCGAGGAATTTGTCCTTGCCGCCCACCTTGTCCCAGTCGATCTTTTC
ATAAAGACCATCGGCGCAACCGAGCGCCAGCTCCTCCGCCTCGACCTGAA
CCACGTCCCAATTCGGCGAGCCGGCCTTCACTTTTGACTGGATGACCCCG
TAACCGCCATCCCATGATTCATCGAGCAGCGGCTTGCCGGTCTTTTCCGC
AAAAGGTTTGAAATAGATTTCGCGCTGCGCATCCTGATAGTTGCCGCCCC
ATGACGCGACGGTAAGGTCGCGCGCGAAGGCGGGGCCGACGGTGAAGGCA
ACGGTAGCGAAAATTCCGCCCATAAGGGCAAGGCGAGCAAACTTCAT
SEQ. ID no. 6:
nucleic acid sequence encoding “PotD truncate”-
972 bp
CGCGACCTTACCGTCGCGTCATGGGGCGGCAACTATCAGGATGCGCAGCG
CGAAATCTATTTCAAACCTTTTGCGGAAAAGACCGGCAAGCCGCTGCTCG
ATGAATCATGGGATGGCGGTTACGGGGTCATCCAGTCAAAAGTGAAGGCC
GGCTCGCCGAATTGGGACGTGGTTCAGGTCGAGGCGGAGGAGCTGGCGCT
CGGTTGCGCCGATGGTCTTTATGAAAAGATCGACTGGGACAAGGTGGGCG
GCAAGGACAAATTCCTCGACAGTGCGGTCAATGATTGCGGGGTTGGCGCC
ATCGTCTGGTCCACTGCCATAGCTTATAATGGCGACAAGCTGAAGGATGG
GCCGAAATCCTGGGCGGATTTCTGGGATGTGAAGAAGTTTCCGGGCAAGC
GATCGCTGCGCAAGAGTGCGAAATACACGCTGGAATTCGCGCTTATGGCT
GATGGCGTCGACAAGGATGACGTTTATGACGTCCTTTCCACGCCGGAAGG
CGTGGACCGCGCCTTCAAGAAGCTCGATGAGCTGAAGCCACATATCGTGT
GGTGGGAAGCCGGTGCGCAGCCCTTGCAATTGCTGGCGTCGGACGAGGTG
GTGATGGCATCGGCTTATAATGGCCGCATCACTGGCATCAACCGTTCCGA
AGGCAAGAATTTCAAGGTCGTCTGGCCGGGCAGCATCTATGCGGTGGATA
GCTGGGTGATTCTGAAGGGGGCTGAAAATAAGGATGCGGGCCTGGATTTC
ATTGCCTTTGCCAGTGAGCCCGAACATCAGGTCAAGCTGCCGAAATATGT
TGCCTACGGCCTGCCGAACAAGGAAGCGGCAGCCAAGGTGCCGGAGGAAT
ATGCAGCAGACCTGCCCACGGCCAAAGCAAACATGAAGGATGCCCTCGCG
CTCGATGTTGATTTCTGGATCGACCATTCAGAAGAGCTGACGAAGCGTTT
CAACGCCTGGCTGGCGCAATAA
SEQ. ID no. 7:
nucleic acid sequence encoding “PotF” protein-
1104 bp
ATGGGGATCAAATCCTTCCTTCTGGCAACGACCGTTGCGACGGGTTTTGT
CGCGGCTGCCACATTTTCCGCAGGCGCGCAGGAGCGGGTGGTCAATATCT
ATAACTGGTCGGATTATATCGACGATTCCATCCTCAAGGACTTCACCAAG
GAGACCGGGATCAAGGTCGTCTACGACGTCTATGACTCCAACGAAATTCT
GGAAACCAAGCTTCTGGCGGGCGGCAGCGGCTACGACCTCGTGGTGCCAT
CGGGCGAATTTCTTGGGCGCCAGATTCCCGCAGGCGTGTTCCTGAAACTC
GACAAGGACAAGCTGCCGAACCTCAAGAATATGTGGGATGAGATTTCGAC
CCGTGCGGCAACCTATGATCCGGGCAACGAATATTCCGTCAATTACATGT
GGGGCACGACCGGCATCGGCTACAATAAGGCCAAGATCAAGGAAGCGCTC
GGCACCGACACGATCGACTCCTGGGACGTGCTTTTCGATCCGGAAAAAAC
GGCAAAGCTGAAAGATTGCGGCATTTACCTGCTTGATTCCGCCAGCGAAA
TGCTGCGTCCGGCGCTGAACTATCTGGGTCTCGACCCGAACTCTCCGTCG
CCGGACGATTTGCAGAAGGCACAGGATCTCTATCTCAAGATTCGTCCGAA
TATCCGCAAATTCCACTCGTCGGAATATATCAATGCGCTCGCCAATGGCG
ATATCTGCATGGCTGTCGGCTATTCCGGCGATATTTTCCAGGCCCGCGAC
CGCGCCGAAAAGGCGAAGCAGGGGGTGGAGATCGGCTATTCGATCCCGAA
GGAAGGCGCGCTGATCTGGTTTGACCAGATGGCGATCCCGGCCGATGCCA
AGCATGTGCCGGAGGCGTTGGAATTCATGAATTATATGATGCGCCCGGAG
GTGGCGGCAAAGGCGTCGAACTATGTGTTCTATGCCAATGGCAACAAGGC
GTCGCAGAAATTCATCGACAAGGAAATCCTCGACGACCCGGAAATCTATC
CGTCCGACGAGGTGATGAAGAAGCTGTTCGTGCCGACGCCATATGACACG
AAGACCCAGCGTGTGGTCACACGCGCCTGGACCAAGATCGTCACCGGCCA
GTAA
SEQ. ID no. 8:
nucleic acid sequence encoding “PotF truncate”
protein- 1026 bp
CAGGAGCGGGTGGTCAATATCTATAACTGGTCGGATTATATCGACGATTC
CATCCTCAAGGACTTCACCAAGGAGACCGGGATCAAGGTCGTCTACGACG
TCTATGACTCCAACGAAATTCTGGAAACCAAGCTTCTGGCGGGCGGCAGC
GGCTACGACCTCGTGGTGCCATCGGGCGAATTTCTTGGGCGCCAGATTCC
CGCAGGCGTGTTCCTGAAACTCGACAAGGACAAGCTGCCGAACCTCAAGA
ATATGTGGGATGAGATTTCGACCCGTGCGGCAACCTATGATCCGGGCAAC
GAATATTCCGTCAATTACATGTGGGGCACGACCGGCATCGGCTACAATAA
GGCCAAGATCAAGGAAGCGCTCGGCACCGACACGATCGACTCCTGGGACG
TGCTTTTCGATCCGGAAAAAACGGCAAAGCTGAAAGATTGCGGCATTTAC
CTGCTTGATTCCGCCAGCGAAATGCTGCGTCCGGCGCTGAACTATCTGGG
TCTCGACCCGAACTCTCCGTCGCCGGACGATTTGCAGAAGGCACAGGATC
TCTATCTCAAGATTCGTCCGAATATCCGCAAATTCCACTCGTCGGAATAT
ATCAATGCGCTCGCCAATGGCGATATCTGCATGGCTGTCGGCTATTCCGG
CGATATTTTCCAGGCCCGCGACCGCGCCGAAAAGGCGAAGCAGGGGGTGG
AGATCGGCTATTCGATCCCGAAGGAAGGCGCGCTGATCTGGTTTGACCAG
ATGGCGATCCCGGCCGATGCCAAGCATGTGCCGGAGGCGTTGGAATTCAT
GAATTATATGATGCGCCCGGAGGTGGCGGCAAAGGCGTCGAACTATGTGT
TCTATGCCAATGGCAACAAGGCGTCGCAGAAATTCATCGACAAGGAAATC
CTCGACGACCCGGAAATCTATCCGTCCGACGAGGTGATGAAGAAGCTGTT
CGTGCCGACGCCATATGACACGAAGACCCAGCGTGTGGTCACACGCGCCT
GGACCAAGATCGTCACCGGCCAGTAA

1. A polypeptide selected from the group consisting of BME110923, BMEI0411, a protective fragment of BME110923, a protective fragment of BMEI0411, an immunologically active variant of BME110923, and an immunologically active variant of BMEI0411, wherein the polypeptide provides a protective immune response against Brucella infection when administered to a mammal.

2. The polypeptide of claim 1, wherein the polypeptide has the amino acid sequence of SEQ ID NO:1 or SEQ ID NO:2.

3. The polypeptide of claim 1, wherein the immunologically active variant has at least 90% sequence identity to BMEII0923 or BMEI0411.

4. The claim 1, tide of claim 1, wherein the protective fragment of BME110923 or BMEI0411 has the amino acid sequence of SEQ ID NO:3 or SEQ ID NO:4, respectively.

5. A pharmaceutical composition comprising the polypeptide claim 1, and an adjuvant.

6. A nucleic acid encoding the polypeptide of claim 1.

7. The nucleic acid of claim 6, wherein the nucleic acid has the nucleic acid sequence of SEQ ID NO:5, SEQ ID NO:6, SEQ ID NO:7, or SEQ ID NO:8.

8. A pharmaceutical composition comprising a plasmid vector containing the nucleic acid of claim 6.

9. A host cell transformed to express the polypeptide of claim 1.

10. (canceled)

11. The polypeptide of claim 1, wherein the Brucella infection is Brucella melitensis infection.

12. (canceled)

13. (canceled)

14. An antibody raised against the polypeptide of claim 1.

15. A method of treating or preventing Brucella infection in a mammal comprising administering to the mammal an effective amount of the polypeptide of claim 1.
